(19) United States
(12) Patent Application Publication
(10) Pub. No.: US 2023/0248867 A1
(43) Pub. Date: Aug. 10, 2023

(54) ELECTRONIC SHIFTING DEVICE WITH STERILIZATION FUNCTION AND STERILIZATION CONTROL METHOD USING THE SAME

(71) Applicants: Hyundai Motor Company, Seoul (KR); Kia Corporation, Seoul (KR)

(72) Inventors: Eun Sik Kim, Gwangmyeong (KR); Ki Young Song, Suwon (KR)

(21) Appl. No.: 17/948,514

(22) Filed: Sep. 20, 2022

(30) Foreign Application Priority Data
Feb. 7, 2022 (KR) 10-2022-0015598

(51) Int. Cl. A61L 2/24; F16H 59/08; A61L 2/10

(52) U.S. Cl. CPC A61L 2/24; F16H 59/08; A61L 2/10; F16H 2059/081; A61L 2202/11; A61L 2202/14; A61L 2202/25

(57) ABSTRACT

An electronic shifting device with a sterilization function is provided. The electronic shifting device comprising a spherical mechanism configured to be rotatable relative to a housing installed in an interior of a vehicle, the spherical mechanism having one side at which a shifting unit having a hemispherical shape is provided, and the other side at which a sterilizing unit having a hemispherical shape is provided, a drive mechanism configured to adjust a rotation angle of the spherical mechanism by providing a rotational force to the spherical mechanism, and a controller configured to control the sterilizing unit so that the sterilizing unit performs a sterilization function in a state in which the shifting unit is hidden in the housing and the sterilizing unit is exposed to the interior of the vehicle by a rotation of the spherical mechanism.

### CROSS-REFERENCE TO RELATED APPLICATION(S)

The present application claims, under 35 U.S.C. § 119(a), priority to Korean Patent Application No. 10-2022-0015598, filed Feb. 7, 2022, the disclosure of which is incorporated herein by reference in its entirety.

### BACKGROUND

### Technical Field

Embodiments of the present disclosure relate to an electronic shifting device with a sterilization function and a sterilization control method using the same, in which a sterilization device is applied to the electronic shifting device, thereby improving a layout for installing the sterilization device.

### Description of the Related Art

Recently, in the era of Covid-19, there has been increased awareness of the importance of a function of sterilizing an interior of a vehicle.

Therefore, there have been developed technologies for sterilizing the interiors of vehicles by using ultraviolet rays. There are currently developed various sterilization devices that are configured to irradiate particular positions, such as a center fascia, a console, a lamp, and the like, with ultraviolet rays.

However, because sterilization devices need to be installed in the vehicle, it is difficult to ensure a layout for installing the sterilization device in the vehicle.

In addition, the sterilization device in the related art may sterilize only a particular space disposed adjacent to the sterilization device or provided at a limited location, which degrades a sterilization effect.

The foregoing explained as the background is intended merely to aid in the understanding of the background of the present disclosure, and is not intended to mean that the present disclosure falls within the purview of the existing technologies that are already known to those skilled in the art.

### SUMMARY

The present disclosure is proposed to solve these problems and aims to provide an electronic shifting device with a sterilization function and a sterilization control method using the same, in which a sterilization device is applied to the electronic shifting device, thereby improving a layout for installing the sterilization device.

To achieve the above-mentioned object, an exemplary embodiment of the present disclosure provides an electronic shifting device with a sterilization function, the electronic shifting device comprising: a spherical mechanism installed to be rotatable relative to a housing installed in an interior of a vehicle, the spherical mechanism having one side at which a shifting unit having a hemispherical shape is provided, and the other side at which a sterilizing unit having a hemispherical shape is provided; a drive mechanism configured to adjust a rotation angle of the spherical mechanism by providing a rotational force to the spherical mechanism; and a controller configured to control the sterilizing unit so that the sterilizing unit performs a sterilization function in a state in which the shifting unit is hidden in the housing and the sterilizing unit is exposed to the interior of the vehicle by a rotation of the spherical mechanism.

Sterilization lamps configured to emit sterilization rays may be embedded in the sterilizing unit, and the sterilization lamps may be configured to be turned on or off by the controller.

The sterilization lamps may be embedded along a surface of the sterilizing unit so that the sterilization rays are concentrated at a center of the sterilizing unit.

A reflection unit may be provided in the interior of the vehicle to which the sterilization rays are emitted from the sterilizing unit.

The controller may reciprocatingly rotate the spherical mechanism at a predetermined rotation angle during a process in which the sterilizing unit performs the sterilization function.

The controller may be configured to control the sterilizing unit so as to cause the sterilization unit to not perform the sterilization function in the state in which the sterilizing unit is hidden in the housing and the shifting unit is exposed to the interior of the vehicle.

Another exemplary embodiment of the present disclosure provides a sterilization control method using the electronic shifting device with a sterilization function, the sterilization control method comprising: a turning on/off determination step of determining, by the controller, whether a driver turns off the vehicle when a condition for operating a sterilization mode on the interior of the vehicle is satisfied; and a sterilization step of performing control to perform, by the sterilizing unit, the sterilization function in the state in which the spherical mechanism is rotated such that the shifting unit is hidden in the housing and the sterilizing unit is exposed to the interior of the vehicle when the controller determines that the driver inputs an operation of turning off the vehicle.

The condition for operating the sterilization mode may be satisfied when the sterilization mode is selected in a state in which the vehicle is at a stationary position.

The sterilization step may further comprise determining a signal for locking a door of the vehicle, such that when the signal for locking the door is inputted, the sterilization function is controlled and performed.

In the sterilization step, the spherical mechanism may reciprocatingly rotate at a predetermined rotation angle in the state in which the sterilizing unit is exposed to the interior of the vehicle.

When the determination result in the turning on/off determination step indicates that the driver does not input the operation of turning off the vehicle, a traveling mode state in which the sterilizing unit is hidden in the housing and the shifting unit is exposed to the interior of the vehicle may be controlled to be maintained.

In the traveling mode state, the sterilizing unit may be controlled so as not to perform the sterilization function.

The sterilization control method of may further comprise a hand disinfection step of performing, by the sterilizing unit, a hand disinfection function when a condition for operating a hand disinfection mode is satisfied and the controller rotates the spherical mechanism such that the shifting unit is hidden in the housing and the sterilizing unit is exposed to the interior of the vehicle.

In the hand disinfection mode, sterilization lamps may emit sterilization rays having lower sterilization intensity than the sterilization rays emitted from the sterilization lamps in the sterilization mode.

The condition for operating the hand disinfection mode may be satisfied when the hand disinfection mode is selected in a state in which the vehicle is at a stationary position.

In the hand disinfection step, the sterilizing unit may operate in a state of being directed toward a front side of the vehicle.

According to the present disclosure described above, the sterilizing unit is installed integrally with the electronic shifting device having a spherical shape. Therefore, hardware components required to install the sterilization device may be eliminated, which makes it possible to greatly reduce the cost and weight of the vehicle. In particular, a space required to install the sterilization device is not additionally needed, which makes it possible to greatly improve a layout in an occupant compartment.

Further, the spherical mechanism emits the sterilization rays while continuously rotating forward and rearward, such that the interior of the vehicle is evenly irradiated with the sterilization rays, and the interior of the vehicle is sterilized by the sterilization rays having high sterilization intensity, thereby cleanly sterilizing various places in the interior of the vehicle.

Therefore, the sterilization function is activated in one of the hand disinfection mode and the sterilization mode which are distinguished, such that in the hand disinfection mode, the sterilization effect may be maximized without adversely affecting the human body. In addition, since the function of the hand disinfection mode may be used to easily sterilize the hand, the mobile phone, the bag, and the like in the vehicle without a separate hand disinfectant, thereby greatly improving marketability of the vehicle.

In a further aspect, a vehicle is provided that comprises an electronic shifting device as disclosed herein. For instance, a vehicle is provided that comprises an electronic shifting device comprising: (a) a spherical mechanism configured to be rotatable relative to a housing installed in an interior of a vehicle, the spherical mechanism comprising: (i) one side at which a shifting unit having a hemispherical shape is provided; and (ii) another side at which a sterilizing unit having a hemispherical shape is provided; (b) a drive mechanism configured to adjust a rotation angle of the spherical mechanism by providing a rotational force to the spherical mechanism; and (c) a controller configured to control the sterilizing unit so that the sterilizing unit performs a sterilization function in a state in which the shifting unit is hidden in the housing and the sterilizing unit is exposed to the interior of the vehicle by a rotation of the spherical mechanism.

### BRIEF DESCRIPTION OF THE DRAWINGS

FIG. 1 is a perspective view illustrating a spherical electronic shifting device having an exposed shifting unit according to the present disclosure.

FIG. 2 is a perspective view illustrating the spherical electronic shifting device having an exposed sterilizing unit according to the present disclosure.

FIG. 3 is a projection view of FIG. 2 when viewed from the lateral side.

FIG. 4 is a view illustrating a state in which a sterilizing unit according to the present disclosure sterilizes a rear side of an occupant compartment.

FIG. 5 is a view illustrating a state in which the sterilizing unit according to the present disclosure sterilizes a front side of the occupant compartment.

FIG. 6 is a view illustrating a state in which a reflective plate is installed in an interior of the vehicle according to the present disclosure.

FIG. 7 is a flow chart illustrating a sterilization control process according to the present disclosure.

### DETAILED DESCRIPTION

Specific structural or functional descriptions of embodiments of the present disclosure disclosed in this specification or application are exemplified only for the purpose of explaining the embodiments according to the present disclosure, the embodiments according to the present disclosure may be carried out in various forms, and it should not be interpreted that the present disclosure is limited to the embodiments described in this specification or application.

Because the embodiments according to the present disclosure may be variously changed and may have various forms, specific embodiments will be illustrated in the drawings and described in detail in the present specification or application. However, the descriptions of the specific embodiments are not intended to limit embodiments according to the concept of the present disclosure to the specific embodiments, but it should be understood that the present disclosure covers all modifications, equivalents and alternatives falling within the spirit and technical scope of the present disclosure.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

The terms such as “first” and/or “second” may be used to describe various constituent elements, but these constituent elements should not be limited by these terms. These terms are used only for the purpose of distinguishing one constituent element from other constituent elements. For example, without departing from the scope according to the concept of the present disclosure, the first constituent element may be referred to as the second constituent element, and similarly, the second constituent element may also be referred to as the first constituent element.

When one constituent element is described as being “coupled” or “connected” to another constituent element, it should be understood that one constituent element can be coupled or connected directly to another constituent element, and an intervening constituent element can also be present between the constituent elements. When one constituent element is described as being “coupled directly to” or “connected directly to” another constituent element, it should be understood that no intervening constituent element is present between the constituent elements. Other expressions, that is, “between” and “just between” or “adjacent to” and “directly adjacent to”, for explaining a relationship between constituent elements, should be interpreted in a similar manner.

The terms used in the present specification are used only for the purpose of describing particular embodiments and are not intended to limit the present disclosure. Singular expressions include plural expressions unless clearly described as different meanings in the context. In the present specification, it should be understood the terms “comprises,” “comprising,” “includes,” “including,” “containing,” “has,” “having” and/or other variations thereof are inclusive and therefore specify the presence of stated features, numbers, steps, operations, elements, components, or combinations thereof, but do not preclude the presence or addition of one or more other features, numbers, steps, operations, elements, components, or combinations thereof

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

Unless otherwise defined, all terms used herein, including technical or scientific terms, have the same meaning as commonly understood by those skilled in the art to which the present disclosure pertains. The terms such as those defined in commonly used dictionaries should be interpreted as having meanings consistent with meanings in the context of related technologies and should not be interpreted as ideal or excessively formal meanings unless explicitly defined in the present specification.

Further, the control unit according to the exemplary embodiment of the present disclosure may be implemented by a non-volatile memory (not illustrated) configured to store an algorithm for controlling operations of various constituent elements in a vehicle or store data related to software commands for executing the algorithm, and by a processor (not illustrated) configured to perform the following operations by using the data stored in the corresponding memory. In this case, the memory and the processor may be implemented as separate chips. Alternatively, the memory and the processor may be implemented as a single chip in which the memory and the processor are integrated. The processor may be configured in the form of one or more processors.

In addition, the terms “control unit” or “unit” including “hybrid controller (HCU)” or “vehicle control unit (VCU)” is merely a term widely used to name a control device (controller) for controlling a particular vehicle function but does not mean a generic function unit. For example, the controller may include a communication device configured to communicate with another controller or a sensor to control a corresponding function, a memory configured to store an operating system, a logic command, and input/output information, and one or more processors configured to perform determination, computation, decision, or the like require the control the corresponding function.

Hereinafter, some embodiments of the present disclosure will be described in detail with reference to the exemplary drawings. In the drawings, the same reference numerals will be used throughout to designate the same or equivalent elements. In addition, a detailed description of well-known features or functions will be ruled out in order not to unnecessarily obscure the gist of the present disclosure.

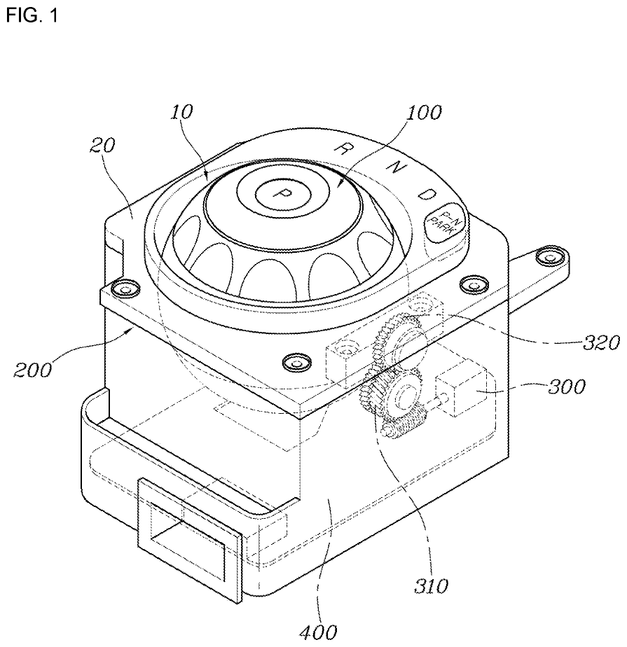

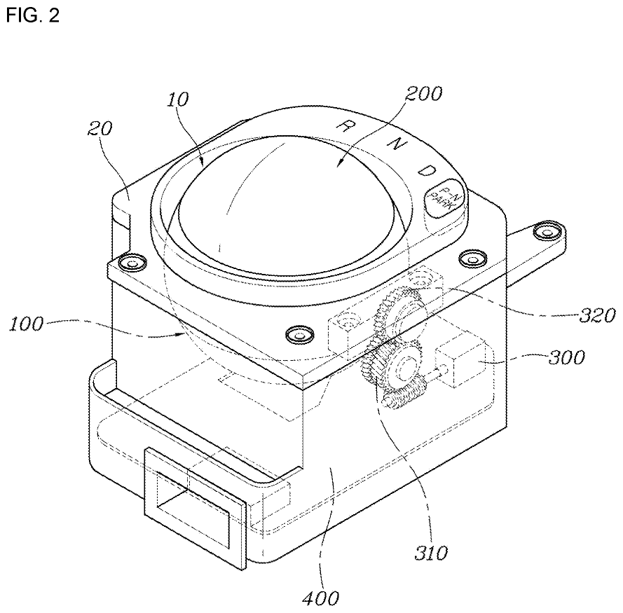

FIG. 1 is a perspective view illustrating a spherical electronic shifting device having an exposed shifting unit 100 according to the present disclosure, and FIG. 2 is a perspective view illustrating the spherical electronic shifting device having an exposed sterilizing unit 200 according to the present disclosure.

Referring to the drawings, an electronic shifting device with a sterilization function according to the present disclosure comprises: a spherical mechanism 10 installed to be rotatable relative to a housing 20 installed in an interior of a vehicle, the spherical mechanism 10 having one side at which the shifting unit 100 having a hemispherical shape is provided, and the other side at which the sterilizing unit 200 having a hemispherical shape is provided; a drive mechanism configured to adjust a rotation angle of the spherical mechanism 10 by providing a rotational force to the spherical mechanism 10; and a controller 400 configured to control the sterilizing unit 200 so that the sterilizing unit 200 performs a sterilization function in a state in which the shifting unit 100 is hidden in the housing 20 and the sterilizing unit 200 is exposed to the interior of the vehicle by a rotation of the spherical mechanism 10.

Specifically, the housing 20 is installed to be positioned at the periphery of a driver seat. The housing 20 is fixedly installed on a console, a center fascia, or the like so that a driver's hand may reach the housing 20.

A rotary shaft is coupled to penetrate a central portion of the spherical mechanism 10. The rotary shaft is installed to be rotatable relative to the housing 20.

Therefore, when the rotary shaft rotates, the spherical mechanism 10 also rotates relative to the housing 20. When the spherical mechanism 10 rotates, one hemispherical side of the spherical mechanism 10 is exposed to the interior of the vehicle through the housing 20, and the other hemispherical side of the spherical mechanism 10 is kept hidden in the housing 20.

The shifting unit 100 is provided at one hemispherical side of the spherical mechanism 10 having a spherical shape, and the sterilizing unit 200 is provided at the other hemispherical side of the spherical mechanism 10.

That is, when the spherical mechanism 10 rotates relative to the housing 20, one of the shifting unit 100 and the sterilizing unit 200 is exposed to the interior of the vehicle, and the other of the shifting unit 100 and the sterilizing unit 200 is kept hidden in the housing 20.

The shifting unit 100 has a shifting mechanism that the driver manipulates to perform a shifting operation.

As illustrated in FIG. 1, the shifting mechanism may be provided in the form of a shifting dial. Although not illustrated in the drawings, the shifting mechanism may be provided in the form of a shifting button or a shifting lever.

In a case in which the shifting mechanism is provided in the form of a shifting dial, a P-position button may be additionally provided.

That is, the driver may be configured to select any one shifting position among a R-position, an N-position, and a D-position by manipulating the shifting dial. The driver may be configured to select the P-position by pushing a separate P-position button.

The drive mechanism comprises a motor 300 fixedly installed in the housing 20, and a plurality of gear members configured to connect the motor 300 and the rotary shaft and transmit power of the motor 300 to the rotary shaft.

The controller 400 controls the operation of the drive mechanism to rotate the spherical mechanism 10 when a signal for operating a hand disinfection mode or a sterilization mode, a turning on/off signal, a signal for locking a door, or the like is inputted to the controller 400.

That is, when a signal for rotating the spherical mechanism 10 is inputted to the controller 400, the motor 300 operates, and driving power of the motor 300 is transmitted to the rotary shaft through a first gear 310 and a second gear 320, such that the rotary shaft rotates relative to the housing 20, and the spherical mechanism 10 rotates together with the rotary shaft.

The rotary shaft of the spherical mechanism 10 rotates by 180 degrees when the drive mechanism operates once. Therefore, when the spherical mechanism 10 rotates, the shifting unit 100 is exposed to the interior of the vehicle through an opening portion of the housing 20 as illustrated in FIG. 1, or the sterilizing unit 200 is exposed to the interior of the vehicle through the opening portion of the housing 20 as illustrated in FIG. 2.

For reference, the controller 400 may have a Hall sensor configured to detect a rotation of the rotary shaft. Therefore, it is possible to more accurately control a rotation angle of the motor 300 by using a signal of the Hall sensor.

Therefore, the controller 400 may be configured to receive an operating signal of the shifting mechanism and output a control signal to a transmission control unit (TCU). The controller 400 may be provided on a PCB provided at a lower end of the spherical mechanism 10 having a spherical shape.

Further, the sterilizing unit 200 is connected to the controller 400 by wiring, such that the sterilizing unit 200 performs a sterilization operation under the control of the controller 400.

For reference, the sterilizing unit 200 may have a light source configured to be turned on or off under the control of the controller 400. A lighting window having a pattern having a particular shape may be provided on a surface of the sterilizing unit 200 so that lighting may be implemented when the light source is turned on.

According to the present disclosure described above, the sterilizing unit 200 is installed integrally with the electronic shifting device having a spherical shape. Therefore, hardware components required to install the sterilization device may be eliminated, which makes it possible to greatly reduce the cost and weight of the vehicle. In particular, a space required to install the sterilization device is not additionally needed, which makes it possible to greatly improve a layout in an occupant compartment.

Further, according to the present disclosure, sterilization lamps 210 configured to emit sterilization rays may be embedded in the sterilizing unit 200, and the sterilization lamps 210 may be configured to be turned on or off by the controller 400.

The sterilization lamps 210 are configured to emit UV sterilization rays. The sterilization lamps 210 may be configured to be connected to the controller 400 by wiring, and the controller 400 may be configured to control an operation of the sterilization lamps 210.

The sterilization lamps 210 are embedded along the surface of the sterilizing unit 200 so that the sterilization rays are concentrated at a center of the sterilizing unit 200.

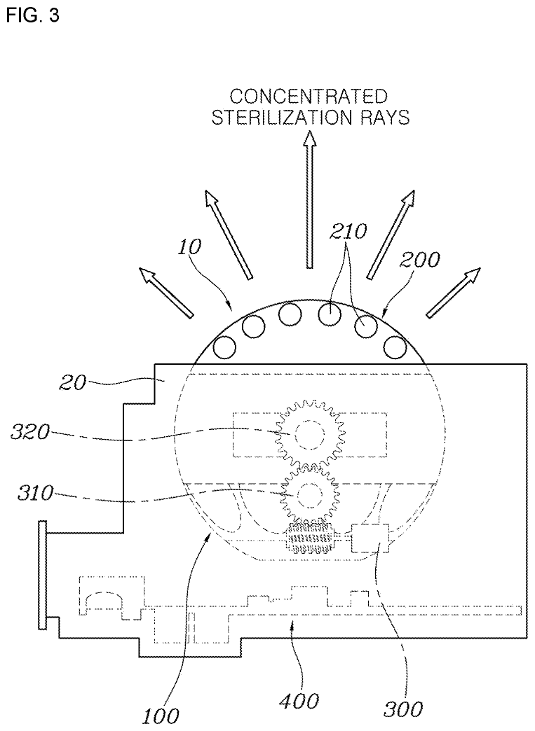

FIG. 3 is a projection view of FIG. 2 when viewed from the lateral side. As illustrated in the drawings, in a case in which the sterilization lamps 210 are installed along an inner surface of the sterilizing unit 200 having a hemispherical shape, the resultant power of components of the sterilization rays emitted from the sterilization lamps 210 is most strongly concentrated at the center of the sterilizing unit 200, such that the sterilization operation is concentratedly performed at the center of the sterilizing unit 200.

Further, according to the present disclosure, a reflection unit 500 may be provided in the interior of the vehicle and reflects the sterilization rays emitted from the sterilizing unit 200.

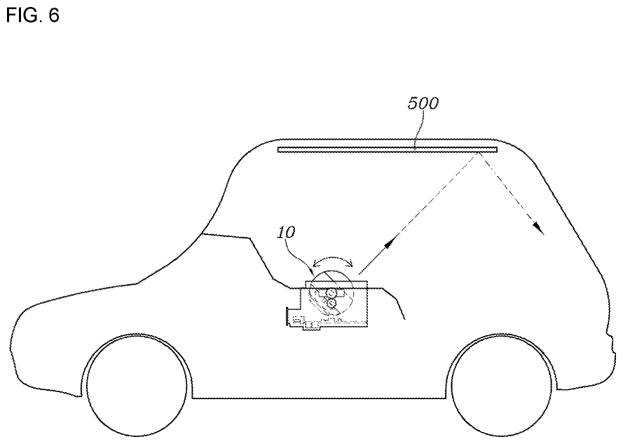

FIG. 6 is a view illustrating a state in which a reflective plate is installed in the interior of the vehicle according to the present disclosure. The reflection unit 500 may be provided in the form of a plate and installed on a ceiling of the interior of the vehicle.

Therefore, when the sterilization rays emitted from the sterilizing unit 200 enter the reflective plate, the sterilization rays entering the reflective plate are reflected at a predetermined reflection angle by the reflective plate in respect to an incident angle of the sterilization rays, such that the sterilization rays are emitted to the interior of the vehicle.

In particular, the controller 400 according to the present disclosure may be configured to reciprocatingly rotate the spherical mechanism 10 by a predetermined rotation angle during the process in which the sterilizing unit 200 performs the sterilization function.

The reciprocating rotation of the spherical mechanism 10 may be repeatedly performed for a predetermined time. The rotation angle may be within a range from an angle at which the sterilizing unit 200 may be configured to concentratedly sterilize a rear seat at a rear side of the occupant compartment to an angle at which the sterilizing unit 200 may be configured to concentratedly sterilize the center fascia at a front side of the occupant compartment.

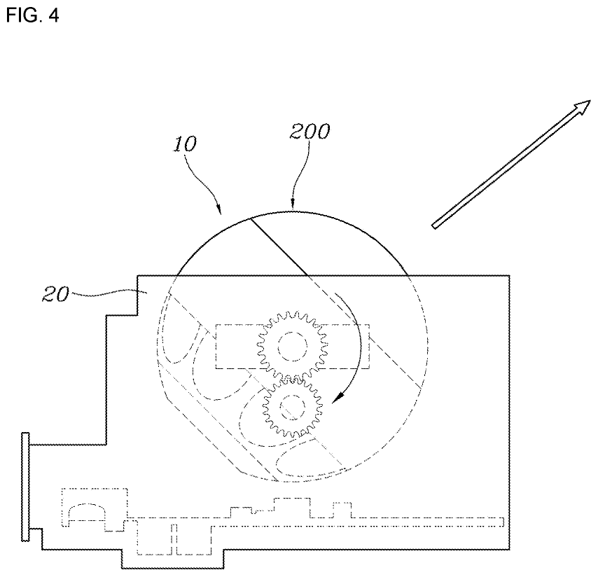

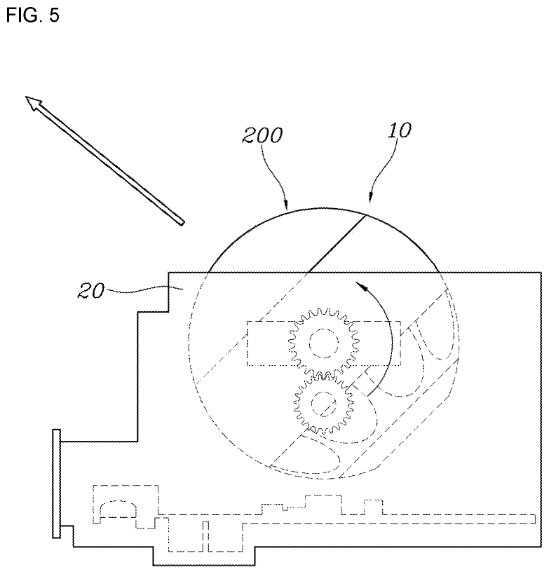

FIG. 4 is a view illustrating a state in which the sterilizing unit 200 according to the present disclosure sterilizes the rear side of an occupant compartment, and FIG. 5 is a view illustrating a state in which the sterilizing unit 200 according to the present disclosure sterilizes the front side of the occupant compartment.

Referring to the drawings, the interior of the vehicle is evenly irradiated with the sterilization rays when the spherical mechanism 10 rotates during the sterilization process. In particular, when the sterilization rays are reflected by the reflective plate, the interior of the vehicle is more evenly irradiated with the sterilization rays, such that the interior of the vehicle may be more cleanly sterilized.

Further, the controller 400 may be configured to perform control so that the sterilizing unit 200 does not perform the sterilization function in the state in which the sterilizing unit 200 is hidden in the housing 20 and the shifting unit 100 is exposed to the interior of the vehicle.

That is, in a traveling situation in which the shifting unit 100 needs to be manipulated like in a traveling mode of the vehicle, the shifting unit 100 is exposed to the outside of the housing 20, and the sterilizing unit 200 is hidden in the housing 20. In this state, the sterilization function is not performed.

Meanwhile, a sterilization control method using the electronic shifting device with a sterilization function according to the present disclosure comprises: a turning on/off determination step of determining, by the controller 400, whether the driver turns off the vehicle when a condition for operating a sterilization mode on the interior of the vehicle is satisfied; and a sterilization step of performing control to perform, by the sterilizing unit 200, the sterilization function in the state in which the spherical mechanism 10 is rotated such that the shifting unit 100 is hidden in the housing 20 and the sterilizing unit 200 is exposed to the interior of the vehicle when the controller 400 determines that the driver inputs the operation of turning off the vehicle.

That is, the sterilization mode is a mode for concentratedly killing bacteria in the vehicle in a state in which the driver is out of the vehicle.

The sterilization mode is performed in the state in which the vehicle is turned off and the sterilizing unit 200 is exposed to the interior of the vehicle. The UV sterilization rays, which are harmful to a human body but have very high sterilization intensity, are emitted from the sterilization lamps 210 and concentratedly sterilize the interior of the vehicle, thereby cleanly sterilizing the interior of the vehicle.

Further, according to the present disclosure, the condition for operating the sterilization mode may be satisfied when the sterilization mode is selected in a state in which the vehicle is at a stationary position.

For example, the stationary position of the vehicle is the P-position or the N-position.

Further, the buttons for selecting the sterilization mode and the hand disinfection mode are provided on the console or the center fascia of the vehicle. Therefore, when the passenger manipulates and pushes the sterilization mode button in the state in which the vehicle is at the P-position or the N-position, the sterilization function is activated.

Therefore, the sterilization step further comprises determining a signal for locking a door of the vehicle, such that when the signal for locking the door is inputted, the sterilization function may be controlled and performed.

For example, the sterilization function is activated after it is determined that the passenger is positioned out of the vehicle even though the condition for operating the sterilization mode is satisfied in the state in which the passenger manipulates the sterilization mode button.

That is, when the door is locked by a locking device such as a smart key within a particular time in the state in which the sterilizing unit 200 is exposed to the interior of the vehicle as the spherical mechanism 10 is rotated by the manipulation of the sterilization mode button, it is determined that the passenger is not present in the interior of the vehicle, and the sterilization function is activated.

In contrast, when the door is not locked within the particular time, it is determined that the passenger is present in the interior of the vehicle, and the sterilization function is not activated.

Further, in the sterilization step, the spherical mechanism 10 may be configured to reciprocatingly rotate at the predetermined rotation angle in the state in which the sterilizing unit 200 is kept exposed to the interior of the vehicle.

That is, the spherical mechanism 10 emits the sterilization rays while continuously rotating forward and rearward, such that the interior of the vehicle is evenly irradiated with the sterilization rays, and the interior of the vehicle is sterilized by the sterilization rays having high sterilization intensity, thereby cleanly sterilizing various places in the interior of the vehicle.

Meanwhile, when the determination result in the turning on/off determination step indicates that the driver does not input the operation of turning off the vehicle, the traveling mode state in which the sterilizing unit 200 is hidden in the housing 20 and the shifting unit 100 is exposed to the interior of the vehicle may be controlled to be maintained.

In particular, in the traveling mode state, the sterilization function may be controlled so as not to be performed by the sterilizing unit 200.

That is, even though the passenger manipulates the sterilization mode button, the sterilization function is not activated in the state in which the vehicle is turned on, thereby preventing the sterilization rays from adversely affecting the passenger.

Meanwhile, the sterilization control method may further comprise a hand disinfection step of performing, by the sterilizing unit 200, a hand disinfection function when the condition for operating the hand disinfection mode is satisfied and the controller 400 rotates the spherical mechanism 10 such that the shifting unit 100 is hidden in the housing 20 and the sterilizing unit 200 is exposed to the interior of the vehicle.

In the hand disinfection mode, the sterilization lamps 210 may be configured to emit sterilization rays having lower sterilization intensity than the sterilization rays emitted from the sterilization lamps 210 in the sterilization mode.

That is, the UV sterilization rays have a high effect of killing harmful bacteria but have wavelengths that adversely affect the human body. Further, the UV sterilization rays have wavelengths that are not harmful to the human body but have slightly low sterilization intensity.

The hand disinfection mode is a mode in which the sterilizing unit 200 sterilizes the driver's hands, a mobile phone, a bag, or the like in a state in which the vehicle is stopped or parked and the driver takes a rest.

Therefore, in the hand disinfection mode, the sterilizing unit 200 sterilizes the driver's belongings by emitting the UV sterilization rays having slightly low sterilization intensity but having wavelengths that are not harmful to the human body.

According to the present disclosure described above, the sterilization function is activated in one of the hand disinfection mode and the sterilization mode which are distinguished, such that particularly in the hand disinfection mode, the sterilization effect may be maximized without adversely affecting the human body.

In addition, since the function of the hand disinfection mode may be used to easily sterilize the hand, the mobile phone, the bag, and the like in the vehicle without a separate hand disinfectant, thereby greatly improving marketability of the vehicle.

Further, according to the present disclosure, the condition for operating the hand disinfection mode may be satisfied when the hand disinfection mode is selected in a state in which the vehicle is at the stationary position.

For example, the stationary position of the vehicle is the P-position or the N-position.

Therefore, when the passenger manipulates and pushes the hand disinfection mode button in the state in which the vehicle is at the P-position or the N-position, the sterilization function according to the hand disinfection mode is activated.

Further, in the hand disinfection step, the sterilizing unit 200 may be configured to operate in a state of being directed toward the front side of the vehicle.

For example, as the sterilizing unit 200 rotates further by 45° (rotation at 225°) toward the front side of the vehicle from a state (rotation at 180°) illustrated in FIG. 3, a central portion of the sterilizing unit 200 is diagonally directed toward the front side of the vehicle.

Therefore, the sterilization rays emitted from the center of the sterilizing unit 200 are not directed directly toward the driver and the occupant. Therefore, it is possible to prevent a risk that the eyesight of the driver and occupant from being damaged by the sterilization rays.

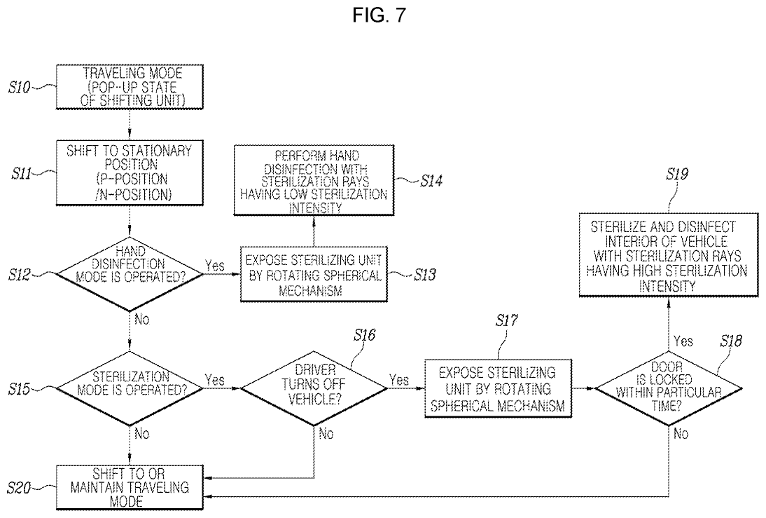

FIG. 7 is a flow chart illustrating a sterilization control process according to the present disclosure.

Hereinafter, the sterilization control process according to the present disclosure will be described with reference to FIG. 7. First, in the traveling mode of the vehicle, the driver operates the shifting unit 100, and the vehicle travels in the state in which the sterilizing unit 200 is hidden in the housing 20, and the shifting unit 100 is exposed to the interior of the vehicle (S10).

In the traveling mode, whether the shifting position is shifted to the P-position or the N-position (S11) and the passenger manipulates the hand disinfection mode button or the sterilization mode button is monitored (S12 and S15).

When the passenger manipulates the hand disinfection mode button during the monitoring process (S12 and S15), the spherical mechanism 10 rotates, such that the sterilizing unit 200 is exposed to the interior of the vehicle, and the shifting unit 100 is hidden in the housing 20 (S13).

Next, the operations of the sterilization lamps 210 are controlled so that the sterilization lamps 210 emit the sterilization rays having low sterilization intensity, and the sterilization rays disinfect the driver's hands or belongings (S14).

Meanwhile, in the monitoring process (S12 and S15), whether the vehicle is turned on or off is determined when the passenger manipulates the sterilization mode button (S16).

When the turning on/off determination result in step S16 indicates that the driver inputs the operation of turning off the vehicle, the spherical mechanism 10 rotates, such that the sterilizing unit 200 is exposed to the interior of the vehicle, the shifting unit 100 is hidden in the housing 20 (S17).

Next, whether the door is locked by a key of the vehicle within a particular time (ex: about 10 minutes) is determined (S18).

When the determination result in step S18 indicates that the door is locked, the operations of the sterilization lamps 210 are controlled so that the sterilization lamps 210 emit the sterilization rays having high sterilization intensity, such that the interior of the vehicle is evenly sterilized and disinfected by the sterilization rays (S19). In this case, the spherical mechanism 10 continuously rotates toward the front side (about 225°) and the rear side (about 135°), such that various places in the interior of the vehicle are more cleanly sterilized and disinfected.

In contrast, when the door is not locked within the particular time, the spherical mechanism 10 rotates, and the mode is shifted to the traveling mode in which the sterilizing unit 200 is hidden in the housing 20 and the shifting unit 100 is exposed to the interior of the vehicle (S20).

In addition, the spherical mechanism 10 is kept in the traveling mode as it is even in the case in which the turning on/off determination result in step S16 indicates that the driver does not input the operation of turning off the vehicle (S20).

Of course, the spherical mechanism 10 is kept in the traveling mode as it is even in the case in which the hand disinfection mode button or the sterilization mode button is not manipulated (S20).

According to the present disclosure described above, the sterilizing unit 200 is installed integrally with the electronic shifting device having a spherical shape. Therefore, hardware components required to install the sterilization device may be eliminated, which makes it possible to greatly reduce the cost and weight of the vehicle. In particular, a space required to install the sterilization device is not additionally needed, which makes it possible to greatly improve a layout in an occupant compartment.

Further, the spherical mechanism 10 emits the sterilization rays while continuously rotating forward and rearward, such that the interior of the vehicle is evenly irradiated with the sterilization rays, and the interior of the vehicle is sterilized by the sterilization rays having high sterilization intensity, thereby cleanly sterilizing various places in the interior of the vehicle.

Therefore, the sterilization function is activated in one of the hand disinfection mode and the sterilization mode which are distinguished, such that particularly in the hand disinfection mode, the sterilization effect may be maximized without adversely affecting the human body. In addition, since the function of the hand disinfection mode may be used to easily sterilize the hand, the mobile phone, the bag, and the like in the vehicle without a separate hand disinfectant, thereby greatly improving marketability of the vehicle.

While the present disclosure has been described with reference to the specific examples, it is apparent to those skilled in the art that various modifications and alterations may be made within the technical spirit of the present disclosure, and these modifications and alterations belong to the appended claims.

What is claimed is:

1. An electronic shifting device with a sterilization function, the electronic shifting device comprising:
a spherical mechanism configured to be rotatable relative to a housing installed in an interior of a vehicle, the spherical mechanism comprising:
one side at which a shifting unit having a hemispherical shape is provided; and
another side at which a sterilizing unit having a hemispherical shape is provided;
a drive mechanism configured to adjust a rotation angle of the spherical mechanism by providing a rotational force to the spherical mechanism; and
a controller configured to control the sterilizing unit so that the sterilizing unit performs a sterilization function in a state in which the shifting unit is hidden in the housing and the sterilizing unit is exposed to the interior of the vehicle by a rotation of the spherical mechanism.

2. The electronic shifting device of claim 1, further comprising sterilization lamps, configured to emit sterilization rays, embedded in the sterilizing unit, wherein the sterilization lamps are configured to be turned on or off by the controller.

3. The electronic shifting device of claim 2, wherein the sterilization lamps are embedded along a surface of the sterilizing unit so that the sterilization rays are concentrated at a center of the sterilizing unit.

4. The electronic shifting device of claim 2, further comprising a reflection unit provided in the interior of the vehicle to which the sterilization rays are emitted from the sterilizing unit.

5. The electronic shifting device of claim 1, wherein the controller is configured to reciprocatingly rotate the spherical mechanism at a predetermined rotation angle during a process in which the sterilizing unit performs the sterilization function.

6. The electronic shifting device of claim 1, wherein the controller is configured to control the sterilizing unit so as to cause the sterilization unit to not perform the sterilization function in the state in which the sterilizing unit is hidden in the housing and the shifting unit is exposed to the interior of the vehicle.

7. A sterilization control method, comprising:
determining, by a controller of an electronic shifting device, whether a driver turns off a vehicle when a condition for operating a sterilization mode on the interior of the vehicle is satisfied, wherein the electronic shifting device comprises:
a spherical mechanism configured to be rotatable relative to a housing installed in an interior of the vehicle, the spherical mechanism comprising:
one side at which a shifting unit having a hemispherical shape is provided; and
another side at which a sterilizing unit having a hemispherical shape is provided;
a drive mechanism configured to adjust a rotation angle of the spherical mechanism by providing a rotational force to the spherical mechanism; and
the controller configured to control the sterilizing unit so that the sterilizing unit performs a sterilization function in a state in which the shifting unit is hidden in the housing and the sterilizing unit is exposed to the interior of the vehicle by a rotation of the spherical mechanism; and
performing, by the sterilizing unit, the sterilization function in a state in which the spherical mechanism is rotated such that the shifting unit is hidden in the housing and the sterilizing unit is exposed to the interior of the vehicle when the controller determines that the driver has input an operation of turning off the vehicle.

8. The sterilization control method of claim 7, wherein the condition for operating the sterilization mode is satisfied when the sterilization mode is selected in a state in which the vehicle is at a stationary position.

9. The sterilization control method of claim 7, wherein performing the sterilization function further comprises determining a signal for locking a door of the vehicle, such that, when the signal for locking the door is inputted, the sterilization function is controlled and performed.

10. The sterilization control method of claim 7, wherein performing the sterilization function further comprises reciprocatingly rotating the spherical mechanism at a predetermined rotation angle in a state in which the sterilizing unit is exposed to the interior of the vehicle.

11. The sterilization control method of claim 7, further comprising, when the determining indicates that the driver does not input the operation of turning off the vehicle, maintaining a traveling mode state in which the sterilizing unit is hidden in the housing and the shifting unit is exposed to the interior of the vehicle.

12. The sterilization control method of claim 11, further comprising, in the traveling mode state, controlling the sterilizing unit so as to not perform the sterilization function.

13. The sterilization control method of claim 7, further comprising performing, by the sterilizing unit, a hand disinfection function when:
a condition for operating a hand disinfection mode is satisfied; and
the controller rotates the spherical mechanism such that the shifting unit is hidden in the housing and the sterilizing unit is exposed to the interior of the vehicle.

14. The sterilization control method of claim 13, wherein performing the hand disinfection function further comprises, emitting, using sterilization lamps, sterilization rays having lower sterilization intensity than sterilization rays emitted from sterilization lamps in the sterilization mode.

15. The sterilization control method of claim 13, wherein the condition for operating the hand disinfection mode is satisfied when the hand disinfection mode is selected in a state in which the vehicle is at a stationary position.

16. The sterilization control method of claim 13, wherein when performing the hand disinfection function, the sterilizing unit operates in a state of being directed toward a front side of the vehicle.

17. A vehicle comprising an electronic shifting device of claim 1.
